(19) United States
(12) Patent Application Publication
(10) Pub. No.: US 2012/0283592 A1
(43) Pub. Date: Nov. 8, 2012

(54) SYSTEM AND METHOD FOR SIMULTANEOUS LUNG FUNCTION ASSESSMENT IN PARALLEL SUBJECTS

(75) Inventors: Thomas Florian Schuessler, Quebec (CA); Anette Robichaud, Quebec (CA); Ilan Benjamin Urovitch, Quebec (CA)

(73) Assignee: Scireq Scientific Respiratory Equipment Inc., Montreal, Quebec (CA)

(21) Appl. No.: 13/505,847

(22) PCT Filed: Nov. 3, 2010
(86) PCT No.: PCT/CA10/01783

§ 371 Date: Jul. 23, 2012

Related U.S. Application Data
(60) Provisional application No. 61/257,641, filed on Nov. 3, 2009.

(51) Int. Cl. A61B 5/097; A61B 5/08; A61M 16/20; A61M 16/10; A61B 5/087; A61M 16/00; A61B 5/113

(52) U.S. Cl. 600/534; 128/204.18; 600/529; 600/543; 128/203.12; 600/538; 128/204.23

(57) ABSTRACT

The present invention relates to a lung function assessment system, a mechanical ventilator and method that allows simultaneously measurements of lung function and provides simultaneously mechanical ventilation to multiple subjects requiring one source of gas.

### FIELD OF THE INVENTION

The present invention generally relates to a lung function assessment system. The present invention more precisely relates to devices for mechanical ventilation and lung function assessment in medical research, specifically systems that allow simultaneous mechanical ventilation and invasive measurement of lung function of multiple parallel subjects requiring one single flow source.

### BACKGROUND OF THE INVENTION

Hundreds of millions of people around the world suffer from respiratory diseases every day. According to the latest World Health Organisation estimates (2007), currently 300 million people have asthma and 210 million people have chronic obstructive pulmonary disease while millions more have allergic rhinitis and other, often underdiagnosed respiratory diseases. Consequently, research into respiratory diseases is a very important and active field.

There is currently a wide variety of documented apparatuses and methods to invasively measure lung function in anaesthetized, mechanically ventilated laboratory animals including, without limitation, mice, rats, guinea pigs, rabbits and primates. To the best of the Applicant's knowledge, all of these methods require one independent hardware setup per subject. Therefore each animal's airway opening is connected to a separate ventilator circuit and raw data are collected through entirely separate sets of transducers for each subject. In addition inhaled aerosol is administered through separate aerosolisation devices for each subject.

One specific technique for measuring lung function in anaesthetized, mechanically ventilated laboratory animals is to calculate the input impedance of the respiratory system from short finite data sets collected when mechanical ventilation is briefly suspended and a predetermined flow, volume or pressure waveform is imposed by a suitable device onto the subject's airway opening. Depending on the exact nature of the desired measurement, this waveform may contain one single frequency or a broader mix of frequencies. This approach is commonly referred to as the Forced Oscillation Technique (FOT). To the best of the Applicant's knowledge, all FOT systems produced or proposed to date require one oscillator device for each subject.

Requiring independent ventilator and/or oscillator systems for each subject significantly limits the control over scientific protocols and the efficiency of experimentation. Consequently, researchers are presently forced to choose between the following options, each of which has its distinct disadvantages:
1. Studying subjects in series on a single device often requires several days to complete experimentation on all subjects, which may lead to increased variability in the resulting data. Variability can be caused, for example, by the natural physiologic daily cycle of the subjects when measurements are obtained at different times of the day. In multi-day experiments, variability can also be caused, for example, by imperfect reproduction of actions such as system calibration, anaesthesia, compound preparation, or by deterioration of pharmacological compounds with time. Moreover, studying subjects in series is ill-suited for studies that require many subjects to be measured in a short time frame, e.g. studying a litter of cubs at a fixed time after birth or studying a large group of subjects at a fixed time after exposure to an inhaled toxin.
2. Studying subjects in parallel on independent parallel devices accelerates the execution of protocols and permits some control over time-of-day and day-to-day variability. However, this approach is subject to potential variability between the systems or components thereof, including, without limitation, the documented inherent variability between individual nebulisation devices of the same type. This approach also involves comparatively high initial equipment cost and operating expenditures.
3. Reverting to simpler, less invasive techniques such as double-chamber plethysmography (DCP) or unrestrained whole-body plethysmography (UWP) permits higher throughput at comparatively lower cost. However measurements provided by these techniques are generally less accurate, less detailed and less reproducible leading to greater variability and poorer statistical separation of the study groups. Scientific publications demonstrate that some of these non-invasive techniques can falsely detect or completely miss the effects of and intervention due to lack of sensitivity and specificity.

Consequently, there is a need for an improved system and method for simultaneous lung function assessment in multiple subjects.

### SUMMARY OF THE INVENTION

An object of the present invention is to propose a lung function assessment system and method that satisfies at least one of the above-mentioned needs.

An object of the present invention is to provide an apparatus for providing mechanical ventilation to at least two subjects, comprising:
one controllable flow source forcing gas through a conduit;
at least two subject sites disposed in parallel, each site being adapted to accommodate one subject;
at least two cannulae, each cannula being insertable into an airway opening of one subject;
at least two Y-conduits having each a first end, a second end and a stem, the stem being connectable to each cannula;
at least two symmetrical inspiratory conduits having each a first end and a second end, the first ends being connectable to the flow source and the second ends being connected to the first end of each Y-conduit to allow gas from the flow source to be delivered through the cannula to the subject; and
at least two expiratory conduits having each a first end and a second end, the first end of each expiratory conduit being connected to the second end of the Y-conduit and each expiratory conduits having an expiratory valve connected thereto moveable between a closed and an opened position allowing gas to be exhaled through the cannula by the subject.

Another aspect of the invention is to provide a method for providing mechanical ventilation to subjects comprising the steps of:
a) supplying gas from a flow source;
b) delivering gas from the flow source to at least two subjects being disposed in parallel through at least two symmetrical inspiratory conduits, each symmetrical conduit being connected to one subject;
c) activating at least two expiratory valves to open at least two expiratory conduits connectable to the subjects;
d) repeating steps b) and c) for a period of time.

Another aspect of the invention is to provide a method for assessment of lung function comprising the steps of:
a) providing an apparatus comprising:
one controllable flow source forcing gas through a conduit;
at least two subject sites disposed in parallel, each site being adapted to accommodate one subject;
at least two cannulae, each cannula being insertable into an airway opening of one subject;
at least two Y-conduits having each a first end, a second end and a stem, the stem being connectable to each cannula;
at least two symmetrical inspiratory conduits having each a first end and a second end, the first ends being connectable to the flow source and the second ends being connected to the first end of each Y-conduit to allow gas from the flow source to be delivered through the cannula to the subject;
at least two expiratory conduits having each a first end and a second end, the first end of each expiratory conduit being connected to the second end of the Y-conduit and each expiratory conduits having an expiratory valve connected thereto moveable between a closed and an opened position allowing gas to be exhaled through the cannula by the subject;
at least two pulmonary ventilation measuring devices, each being connected to a corresponding subject site; and
a common inspiratory pressure transducer positioned at a branch point between the inspiratory conduits.
b) performing a calibration manoeuvre to characterize each inspiratory pathway, said pathway comprising the inspiratory conduit, the first end of the Y-conduit and the cannula, by providing oscillatory gas flow from the controllable flow source to at least two subject sites, said calibration measurement comprising the steps of
   b1) measuring pressure at a branching point between the inspiratory conduits throughout oscillation;
   b2) measuring individual flows at the subject sites with the ventilation measuring devices throughout oscillation;
c) calculating calibration impedances for each inspiratory pathway as a frequency domain ratio of the pressure at the branching point over the corresponding flow at the subject site.
d) populating the subject sites with subjects
e) performing a measurement manoeuvre by providing oscillatory gas flow from the controllable flow source to at least two subject sites, said measurement comprising the steps of
   e1) measuring pressure at a branching point between the inspiratory conduits throughout oscillation;
   e2) measuring individual flows at the subject sites with the ventilation measuring devices throughout oscillation;
f) calculating individual impedances for each subject according to the following formula:

$${Z}_{tr , k} = \frac{{P}_{insp}}{{\dot{V}}_{k}} - {Z}_{calk}$$

wherein Z_{tr,k} is a transfer impedance of the subject at site k, Pᵢₙₛₚ is a pressure at the branching point, Vₖ is a calibration flow obtained from the flow measurement device at site k and Z_{cal,k} is a calibration impedance of a given pathway.

A non-restrictive description of preferred embodiments of the invention will now be given with reference to the appended drawings.

### BRIEF DESCRIPTION OF THE DRAWINGS

FIG. 1 is a schematic view of a system according to a preferred embodiment of the present invention;

FIG. 2 is a schematic view of an electrical equivalent circuit of system dynamics models for calibration (a) and measurement (b) for the system shown in FIG. 1;

FIG. 3 includes graphs illustrating the response to inhaled MCh obtained from input impedance from conventional FOT and transfer impedance using the method according to a preferred embodiment of the present invention;

FIG. 4 includes graphs illustrating the response to inhaled MCh obtained by parallel transfer impedance using two parallel measurement sites with the method according to a preferred embodiment of the present invention; and

FIG. 5 includes graphs illustrating the real part (R) and imaginary part (X) of the parallel transfer impedance at baseline using two parallel measurement sites with the method according to a preferred embodiment of the present invention.

### DETAILED DESCRIPTION OF PREFERRED EMBODIMENTS OF THE INVENTION

The apparatus of the present invention assess lung function and/or provide mechanical ventilation to many subjects requiring one flow source. The subjects of the present invention include rodents, primates, canines, felines, ovines and bovines. The subjects are disposed in parallel and are connected to a common flow source through symmetrical conduits. Symmetrical conduits have the same mechanical properties allowing a precise and a reproducible assessment of the lung function and/or provide a reproducible mechanical ventilation to the different subjects.

A aspect of the present invention is to provide an apparatus for providing mechanical ventilation to at least two subjects, comprising:
one controllable flow source forcing gas through a conduit;
at least two subject sites disposed in parallel, each site being adapted to accommodate one subject;
at least two cannulae, each cannula being insertable into an airway opening of one subject;
at least two Y-conduits having each a first end, a second end and a stem, the stem being connectable to each cannula;
at least two symmetrical inspiratory conduits having each a first end and a second end, the first ends being connectable to the flow source and the second ends being connected to the first end of each Y-conduit to allow gas from the flow source to be delivered through the cannula to the subject; and
at least two expiratory conduits having each a first end and a second end, the first end of each expiratory conduit being connected to the second end of the Y-conduit and each expiratory conduits having an expiratory valve connected thereto moveable between a closed and an opened position allowing gas to be exhaled through the cannula by the subject.

Preferably the apparatus further comprises at least two pulmonary ventilation measuring devices, each being connected to a corresponding subject site.

Preferably the pulmonary ventilation measurement devices comprise chest wall movement measurement devices.

Preferably each chest wall movement measurement device comprises a closed chamber containing a single port to atmosphere fitted with a flow sensor measuring flow into and out of said chambers.

Preferably the apparatus further comprises at least two inspiratory valves, each inspiratory valve being integrated into a corresponding inspiratory conduit.

Preferably the apparatus further comprises a nebulizer connected downstream from the flow source to enrich the gas with an aerosol before supplying the gas within the inspiratory conduits.

Preferably the aerosol is methacholine, acetylcholine, ovalbumine, histamine, saline, carbachol or a pharmacological bronchodilator.

Preferably the flow source comprises a piston connected to a gas source, the piston injecting the gas into the inspiratory conduits.

Preferably the flow source further comprises a central inspiratory valve and an intake valve connected to the piston.

Preferably the apparatus further comprises a common inspiratory pressure transducer downstream from the flow source to measure the pressure within the inspiratory conduits.

Preferably the transducer is positioned at a branch point between the inspiratory conduits.

Preferably the expiratory conduits are symmetrical and the second ends of the expiratory conduits are connected via an expiratory manifold to a device for applying positive end-expiratory pressure.

Preferably the device for applying positive end-expiratory pressure comprises:
a proportional valve:
an expiratory pressure transducer to measure pressure within the expiratory manifold; and
a controller for maintaining a constant positive end-expiratory pressure within the expiratory manifold throughout an expiratory phase by controlling the proportional valve.

The present invention also provides a method for providing mechanical ventilation to subjects comprising the steps of:
a) supplying gas from a flow source;
b) delivering gas from the flow source to at least two subjects being disposed in parallel through at least two symmetrical inspiratory conduits, each symmetrical conduit being connected to one subject;
c) activating at least two expiratory valves to open at least two expiratory conduits connectable to the subjects;
d) repeating steps b) and c) for a period of time.

The method preferably comprises, prior to step d) the steps of:
C1) measuring an end-expiratory pressure within the expiratory conduits through a pressure transducer connected to the at least two expiratory conduits;
C2) maintaining a constant positive and expiratory pressure within the expiratory conduits upon activation of the expiratory valves and through control of a proportional valve connecting the at least two expiratory conduits together.

The method preferably further comprises the steps of adjusting at least two inspiratory valves, each valve being connected to a corresponding symmetrical inspiratory conduit and each valve being controlled to allow equal tidal volume to be delivered to the subjects.

The present invention also provides a method for assessment of lung function comprising the steps of:
a) providing an apparatus comprising:
one controllable flow source forcing gas through a conduit;
at least two subject sites disposed in parallel, each site being adapted to accommodate one subject;
at least two cannulae, each cannula being insertable into an airway opening of one subject;
at least two Y-conduits having each a first end, a second end and a stem, the stem being connectable to each cannula;
at least two symmetrical inspiratory conduits having each a first end and a second end, the first ends being connectable to the flow source and the second ends being connected to the first end of each Y-conduit to allow gas from the flow source to be delivered through the cannula to the subject;
at least two expiratory conduits having each a first end and a second end, the first end of each expiratory conduit being connected to the second end of the Y-conduit and each expiratory conduits having an expiratory valve connected thereto moveable between a closed and an opened position allowing gas to be exhaled through the cannula by the subject;
at least two pulmonary ventilation measuring devices, each being connected to a corresponding subject site; and
a common inspiratory pressure transducer positioned at a branch point between the inspiratory conduits.
b) performing a calibration manoeuvre to characterize each inspiratory pathway, said pathway comprising the inspiratory conduit, the first end of the Y-conduit and the cannula, by providing oscillatory gas flow from the controllable flow source to at least two subject sites, said calibration measurement comprising the steps of
   b1) measuring pressure at a branching point between the inspiratory conduits throughout oscillation;
   b2) measuring individual flows at the subject sites with the ventilation measuring devices throughout oscillation;
c) calculating calibration impedances for each inspiratory pathway as a frequency domain ratio of the pressure at the branching point over the corresponding flow at the subject site.
d) populating the subject sites with subjects
e) performing a measurement manoeuvre by providing oscillatory gas flow from the controllable flow source to at least two subject sites, said measurement comprising the steps of
   e1) measuring pressure at a branching point between the inspiratory conduits throughout oscillation;
   e2) measuring individual flows at the subject sites with the ventilation measuring devices throughout oscillation;
f) calculating individual impedances for each subject according to the following formula:

$${Z}_{tr , k} = \frac{{P}_{insp}}{{\dot{V}}_{k}} - {Z}_{calk}$$

wherein Z_{tr,k} is a transfer impedance of the subject at site k, Pᵢₙₛₚ is a pressure at the branching point, Vₖ is a calibration flow obtained from the flow measurement device at site k and Z_{cal,k} is a calibration impedance of a given pathway.

Preferably the oscillatory gas flow in steps b) and e) is controlled to reproduce a predetermined flow rate, volume or pressure waveform.

Preferably the waveform varies at a single frequency or a broader mix of frequencies.

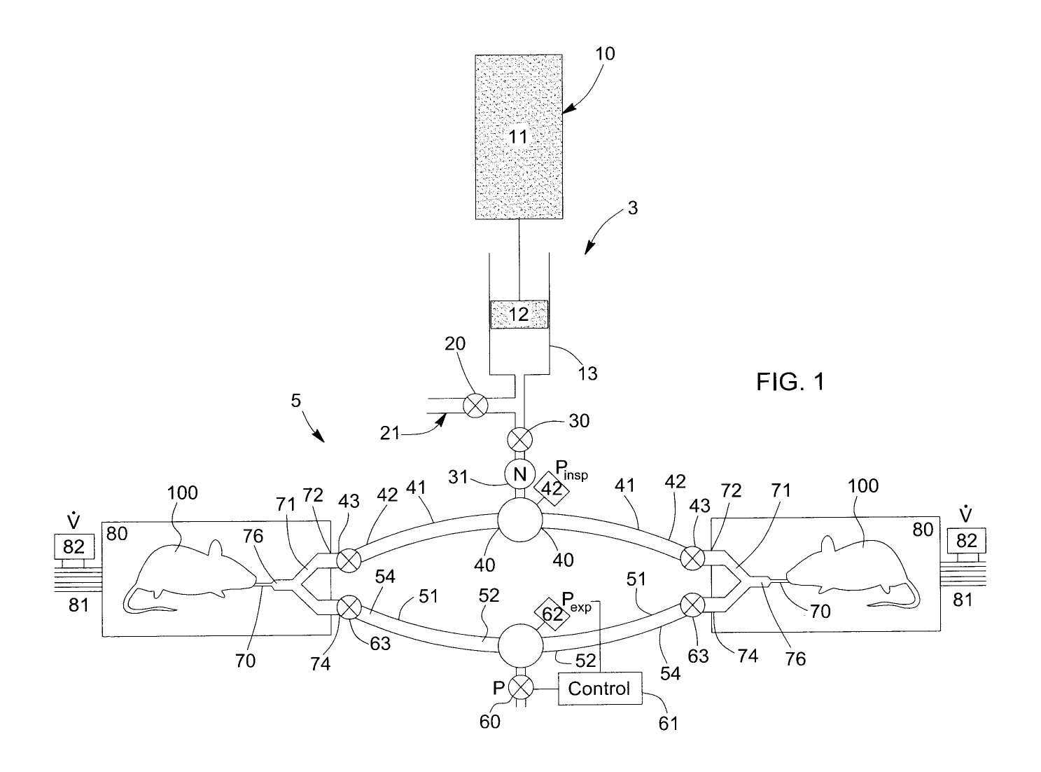

Now referring to FIG. 1, there is shown a mechanical ventilator system 5, wherein at least two subjects 100 are disposed in parallel. A single flow source 3 is provided to apply mechanical ventilation to the subjects. In one embodiment, the flow source 3 provides gas to the subjects, but a person skilled in the art would understand that the flow source can be provided by ambient air. At least two symmetrical inspiratory conduits 41 having the same mechanical properties are connected to the flow source 3. The inspiratory conduits 41 have each a first end 40 and a second end 42. The first ends 40 are connected to the flow source 3 in order for the gas to be delivered into the inspiratory conduits 41. The first ends 40 are connected to a common suitable pressure transducer 42 in order to measure the pressure within the conduits 41 (Pᵢₙₛₚ). The second ends 42 of the inspiratory conduits 41 are each connected to a Y-conduit 71. In one embodiment, individual inspiratory valve 43 is connected to each of the inspiratory conduit 41 to individually control inspiratory flow for each subject. The individual inspiratory valves 43 are moveable between a closed and an opened position allowing equal tidal volume to be delivered to the subjects if such characteristic is desired.

The mechanical ventilator 5 also contains at least two Y-conduits 71, one for each subject. These Y conduits deliver the gas from the inspiratory conduits 41 to the subjects 100. In addition, these Y-conduits allow the exhaled gas to be directed to different conduits than the inhaled gas conduits allowing a control of the exhaled gas pressure. Each Y-conduit 71 has a first end 72, a second end 74 and a stem 76. The first ends 72 are each connected to the second end 42 of the inspiratory conduit 41. The second ends 74 of the Y-conduits 71 are each connected to an expiratory conduit 51, and the stems 76 are each connected to the subject 100 through an intubation or tracheotomy cannula 70. The cannulae 70 are located close to the subject's airway opening to minimize ventilator deadspace.

The mechanical ventilator 5 also contains at least two expiratory conduits 51; one expiratory conduit for each subject. The expiratory conduits 51 are optionally symmetrical, and can have the same mechanical properties. Each expiratory conduit 51 has a first end 52 and a second end 54. The second ends 54 are each connected to the second end 74 of the Y-conduits allowing the exhaled gas from the subjects to be directed into the exhaled conduits 51. The first ends 52 of the expiratory conduits 51 are connected to a common proportional valve 60. A servo-controller 61 is connected to the proportional valve 60 maintaining a constant positive end-expiratory pressure (PEEP) throughout the expiratory phase based on measurement of the pressure in the expiratory conduits 51 (Pₑₓₚ) obtained from a pressure transducer 62 connected to the expiratory conduits 51. Each expiratory conduit 51 contains an individual expiratory valve 63 connected thereto. The expiratory valves 63 avoid a shunt pathway between the airway openings of the individual subjects during the inspiratory phase.

Each subject 100 occupies one subject site for monitoring chest wall displacement.

The mechanical ventilator 5 can be modified in order to assess lung function of multiple subjects requiring one flow source. The subjects are disposed in parallel and are connected to symmetrical inspiratory and expiratory conduits as described above. In order to assess lung function, few elements of the mechanical ventilator 5 are modified. The flow source 3 supplies gas to the system 5, according to the technique the operator intends to use such as FOT manoeuvres. In one embodiment, the flow source comprises an air intake 21 controlled by an intake valve 20. Upon activating the valve 20, a cylinder 13 connected to the air intake 21, is refilled with fresh gas. A computer-controlled piston pump 10, consisting of a linear actuator 11 drives a piston 12 into the cylinder 13. The flow source also contains a central inspiratory valve 30 controlling the gas entry into the system 5 upon compressing the gas into the cylinder 13. The parameters (predetermined flow, volume or pressure waveform, single frequency or a broader mix of frequencies) of the gas to be injected into the system 5 depend on the techniques that the operator intends to use. A person skilled in the art would know these parameters and would also appreciate that any other known controllable flow source is suitable to achieve the same purpose.

Chest wall displacement is measured for each subject 100 at a corresponding subject site when assessing lung functions. In a preferred embodiment, each subject 100 is placed inside an individual body plethysmograph 80. To acquire individual flow data, each body plethysmograph 80 is connected to a flow sensor 81 and a differential pressure transducer 82. In one embodiment, the flow sensor 81 is a pneumotachograph. The body plethysmograph volume and the pneumotachograph resistance are selected to ensure a flat frequency response to sufficiently high frequencies so that the measured flow in and out of the body plethysmograph (\dot{V}) provides a valid and accurate estimate chest wall displacement.

In one embodiment, a nebulizer 31 is connected to the flow source to enrich the gas with an aerosol prior to be injected into the inspiratory conduits. Any suitable aerosols can be used such as methacholine, histamine, saline, carbachol and achethylcholine.

Providing mechanical ventilation to the subjects is performed as follows. A gas is first supplied from the flow source and provided to the subjects disposed in parallel. The gas flows into the symmetrical inspiratory conduits connecting the subjects to the flow source. The inspiratory conduits 41 being symmetric, the differences between the individual inspiratory flow pathways are negligible and the relative tidal volumes delivered to the individual subjects depend solely on their relative lung mechanics. If all subjects have identical lung mechanics, they will receive identical tidal volumes. However, if half of the subjects have lungs twice as stiff as the other half, they will receive only half the tidal volume. In such inhomogeneous circumstances, an intelligent computer controlling the individual inspiratory valves 43 can be used to shorten the inspiration for more compliant subjects, permitting equal tidal volumes to be delivered to an inhomogeneous group of subjects.

At the end of the inspiratory cycle, the gas is then exhaled from the subjects 100, flows into the cannulae 70, the stems 76 of the Y-conduits 71 and the second ends 74 of the Y-conduits and to the expiratory conduits 51. The proportional valve 60 opens as necessary to bring the pressure in the expiratory conduits 51 to the desired PEEP level, and then modulates its degree of opening to maintain the PEEP level throughout the expiration phase. In one embodiment, the intake valve 20 then opens and the piston 12 retracts to refill the cylinder 13 with fresh gas and prepare the next inspiratory phase in order to repeat the cycle for a period of time desired. In one embodiment, inspiratory valves 43 connected to the inspiratory conduits 41 are closed at the end of the inspiratory cycle or the inspiratory valves 43 are adjusted in order to provide equal tidal volume to be delivered to the subjects.

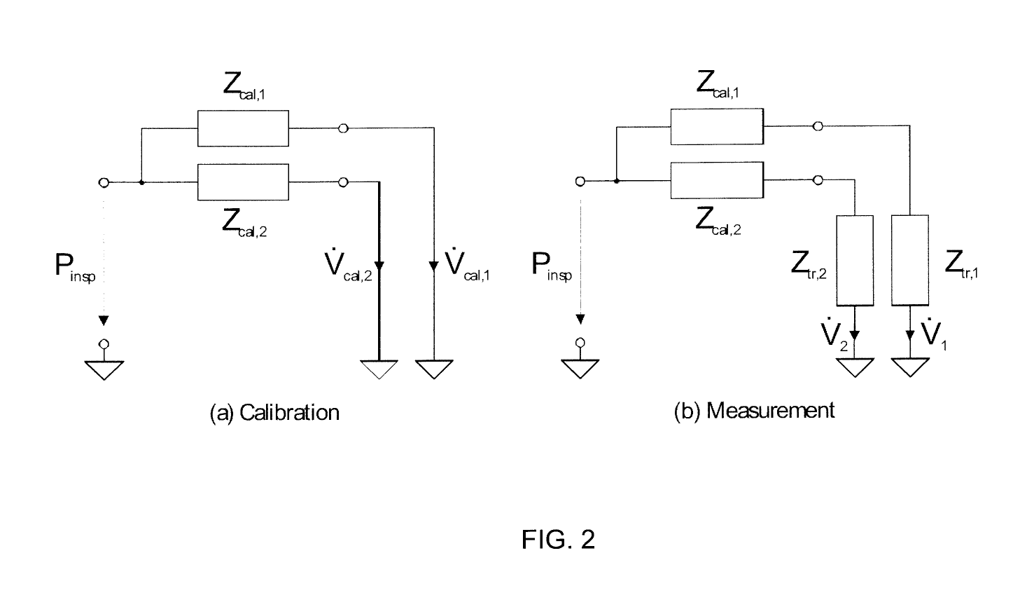

Measuring lung function requires knowledge of the pressure drop across the respiratory system of each subject. In preparation for such measurements, a dynamic calibration manoeuvre is performed at the onset of any given experiment, to individually characterize each inspiratory pathway, including the cannulae 70. During the calibration manoeuvre, the system is assembled with the chambers 80 closed, except the subjects 100 are not connected to the cannulae 70. The individual expiratory valves 63 are closed throughout the calibration manoeuvre in order for the system dynamics to be modelled according to the electrical equivalent circuit shown in FIG. 2(a). Provided that the pneumotachograph resistance is negligible compared to the resistance of the inspiratory pathway defined by the inspiratory conduits 41 and cannulae 70, the calibration impedance of any given pathway k (Z_{cal,k}, 90) from Pᵢₙₛₚ and the calibration flow obtained from the corresponding plethysmograph (\dot{V}_{calk}) can be calculated according to the following formula:

$${Z}_{calk} = \frac{{P}_{insp}}{{\dot{V}}_{calk}} .$$

For any further recordings obtained with the subjects 100 connected to the cannulae 70 throughout the remainder of the experiment, the system dynamics can be modelled according to FIG. 2(b), and the transfer impedance of the respiratory system of subject k (Z_{tr,k}) can be calculated according to the following formula:

$${Z}_{calk} + {Z}_{tr , k} = \frac{{P}_{insp}}{{\dot{V}}_{k}} ,$$

which is easily rearranged to

$${Z}_{tr , k} = \frac{{P}_{insp}}{{\dot{V}}_{k}} - {Z}_{calk} .$$

Depending on the application, parametric models of respiratory mechanics can also be used to represent these data in a more condensed format.

Once the calibration impedances are measured lung function assessment may be carried out during mechanical ventilation. In this case, the steps associated with the method for assessment of lung function are carried out except that the measurement manoeuvre step is replaced with a series of steps to record and segment pressure and flow data obtained during mechanical ventilation.

Although FIG. 1 shows only two parallel subjects, the concept described above is easily extended to more parallel subjects without departing from the scope or spirit of the present invention.

The mechanical ventilator and the lung function system of the present invention provides simultaneous mechanical ventilation and simultaneous measurement of lung function of many subjects. Assessing lung function and providing mechanical ventilation to many subjects simultaneously allow researchers to study a greater numbers of subjects in a shorter period of time. In addition, many subjects can be studied at the same time preventing physiological daily cycle variability of the subjects and variability from different systems. Therefore, the system of the present invention allows a more accurate comparison between the results obtained from the different subjects studied.

### Examples

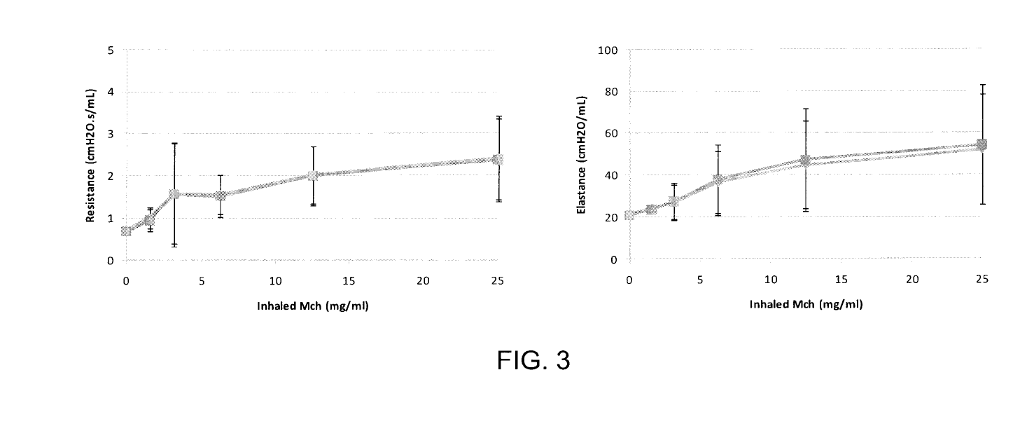

Preliminary validation experiments were carried out using a group of four naïve A/J mice. In a first set of measurements, respiratory mechanics of individual mice in response to inhaled methacholine (MCh) challenge were captured simultaneously by transfer impedance according to the system of the present invention (♦) and input impedance obtained from conventional FOT (▪) as shown in FIG. 3. Both techniques produced virtually identical results.

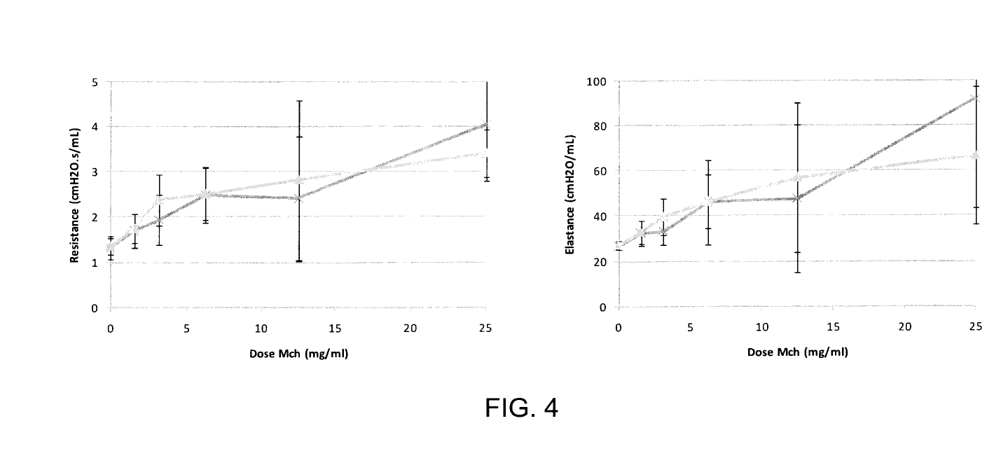

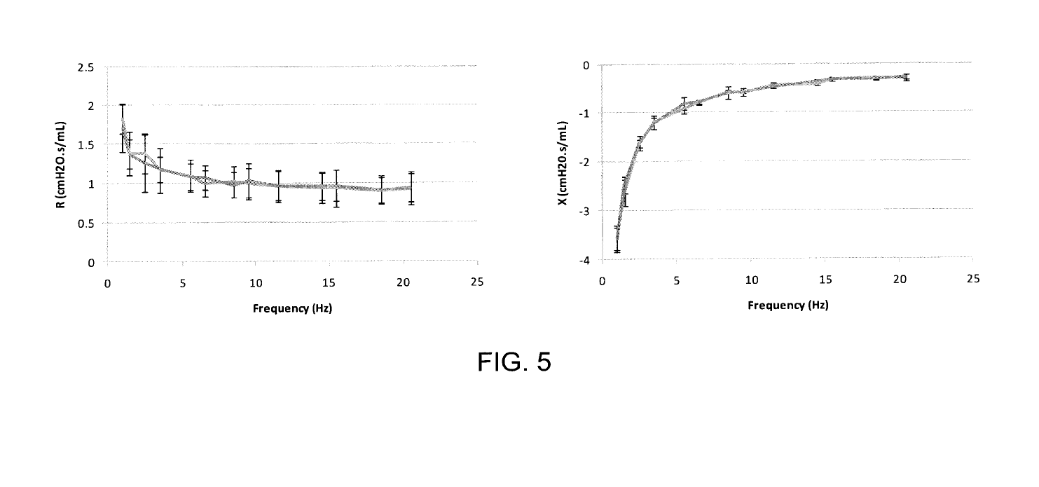

In a second set of measurements, the MCh dose response of eight naïve A/J mice was measured using the system of the present invention with two parallel measurement sites, i.e. by measuring consecutive sets of two parallel mice, where mechanical ventilation, forced oscillation waveforms and aerosol were all provided by a single device for each set of two animals. The data from these animals were grouped by the measurement site on which a subject was placed during recording, resulting in four animals per group. As shown in FIG. 4, both groups showed no significant differences from each other, and the results were comparable to those obtained in individual animals (FIG. 3). Complete transfer impedances obtained from the baseline recordings of each group are shown in FIG. 5.

All subjects appeared adequately ventilated throughout their stay on the device, and no animal showed any signs of discoloration of mucosal membranes or other indications of insufficient gas exchange. The variability between subjects and groups is comparable to the normal physiological variability that is commonly observed in lung function studies. These data show that both mechanical ventilation and nebulized aerosol challenges were adequately distributed to parallel subjects.

In summary, these data demonstrate that the system of the invention permits efficient and accurate mechanical ventilation, aerosol administration and measurement of lung function by means of measuring transfer impedance in parallel subjects with a single gas supply system such as a piston pump and aerosol generator.

Although preferred embodiments of the present invention have been described in detailed herein and illustrated in the accompanying drawings, it is to be understood that the invention is not limited to these precise embodiments and that various changes and modifications may be effected therein without departing from the scope or spirit of the present invention.

1-20. (canceled)

21. An apparatus for providing mechanical ventilation to at least two subjects, comprising:
one controllable flow source forcing a flow of gas through a conduit; the flow of gas having a flow waveform, said flow waveform comprising a combination of a mechanical ventilation waveform and a forced oscillation measurement waveform;
at least two subject sites disposed in parallel, each site being adapted to accommodate one subject;
at least two cannulae, each cannula being insertable into an airway opening of one subject;
at least two Y-conduits having each a first end, a second end and a stem, the stem being connectable to each cannula;
at least two symmetrical inspiratory conduits having each a first end and a second end, the first ends being connectable to the flow source and the second ends being connected to the first end of each Y-conduit to allow gas from the flow source to be delivered through the cannula to the subject; and
at least two expiratory conduits having each a first end and a second end, the first end of each expiratory conduit being connected to the second end of the Y-conduit and each expiratory conduits having an expiratory valve connected thereto moveable between a closed and an opened position allowing gas to be exhaled through the cannula by the subject.

22. The apparatus according to claim 21, further comprising at least two pulmonary ventilation measuring devices, each being connected to a corresponding subject site.

23. The apparatus according to claim 22, wherein the pulmonary ventilation measurement devices comprise chest wall movement measurement devices.

24. The apparatus according to claim 23, wherein each chest wall movement measurement device comprises a closed chamber containing a single port to atmosphere fitted with a flow sensor measuring flow into and out of said chambers.

25. The apparatus of claim 22, further comprising at least two inspiratory valves, each inspiratory valve being integrated into a corresponding inspiratory conduit.

26. The apparatus of claim 22, further comprising a nebulizer connected downstream from the flow source to enrich the gas with an aerosol before supplying the gas within the inspiratory conduits.

27. The apparatus of claim 26, wherein the aerosol is methacholine, acetylcholine, ovalbumine, histamine, saline, carbachol or a pharmacological bronchodilator.

28. The apparatus of claim 22, wherein the flow source comprises a piston connected to a gas source, the piston injecting the gas into the inspiratory conduits.

29. The apparatus of claim 28, wherein the flow source further comprises a central inspiratory valve and an intake valve connected to the piston.

30. The apparatus of claim 32, further comprising a common inspiratory pressure transducer downstream from the flow source to measure the pressure within the inspiratory conduits.

31. The apparatus of claim 30, wherein the transducer is positioned at a branch point between the inspiratory conduits.

32. The apparatus of any one of claim 31, wherein the expiratory conduits are symmetrical and the second ends of the expiratory conduits are connected via an expiratory manifold to a device for applying positive end-expiratory pressure.

33. The apparatus of claim 32, wherein the device for applying positive end-expiratory pressure comprises:
a proportional valve:
an expiratory pressure transducer to measure pressure within the expiratory manifold; and
a controller for maintaining a constant positive end-expiratory pressure within the expiratory manifold throughout an expiratory phase by controlling the proportional valve.

34. The apparatus of claim 33, further comprising a computer comprising:
measurement means for performing a measurement manoeuvre by providing oscillatory gas flow from controllable flow source to the at least two subject sites, said measurement means measuring pressure with the transducer at the branching point between the inspiratory conduits throughout an oscillation and measuring individual flowers at the subject sites within the pulmonary ventilation measuring devices throughout the oscillation; and
a calculator for calculating individual impedances for each subject according to the following formula:
$${Z}_{tr , k} = \frac{{P}_{insp}}{{V}_{k}} - {Z}_{calk}$$
Wherein Z_{tr,k} is a transfer impedance of the subject at site k_{1 P}ᵢₙₛₚ is a pressure at the branching point, Vₖ is a calibration flow obtained from a flow measurement device at site k and Z_{calk} is a calibration impedance of a given pathway.

35. The apparatus of claim 34, wherein the computer further comprises:
calibration means for performing a calibration manoeuvre to characterize each inspiratory pathway, said pathway comprising the inspiratory conduit, the first end of the Y-conduit and the cannula, by providing oscillatory gas flow from the controllable flow source to the at least two subject sites, said calibration means measuring pressure at a branching point between the inspiratory conduits throughout a calibration oscillation, and measuring individual flows at the subject sites with the pulmonary ventilation measuring devices throughout the calibration oscillation;
and wherein the calculator calculates calibration impendences for each inspiratory pathway as a frequency domain ratio of the pressure at the branching point over a corresponding flow at the subject site.

36. The apparatus of claim 34, wherein the oscillatory gas flow is controlled to reproduce one of a predetermined flow rate, volume or pressure waveform.

37. The apparatus of claim 36, wherein the waveform varies at one of a single frequency or a mix of frequencies.

38. A method for assessment of lung function comprising the steps of:
a) providing an apparatus comprising:
one controllable flow source forcing gas through a conduit;
at least two subject sites disposed in parallel, each site being adapted to accommodate one subject;
at least two cannulae, each cannula being insertable into an airway opening of one subject:
at least two Y-conduits having each a first end, a second end and a stem, the stem being connectable to each cannula;
at least two symmetrical inspiratory conduits having each a first end and a second end, the first ends being connectable to the flow source and the second ends being connected to the first end of each Y-conduit to allow gas from the flow source to be delivered through the cannula to the subject;
at least two expiratory conduits having each a first end and a second end, the first end of each expiratory conduit being connected to the second end of the Y-conduit and each expiratory conduits having an expiratory valve connected thereto moveable between a closed and an opened position allowing gas to be exhaled through the cannula by the subject;
at least two pulmonary ventilation measuring devices, each being connected to a corresponding subject site; and
a common inspiratory pressure transducer positioned at a branch point between the inspiratory conduits.
b) performing a calibration manoeuvre to characterize each inspiratory pathway, said pathway comprising the inspiratory conduit, the first end of the Y-conduit and the cannula, by providing oscillatory gas flow from the controllable flow source to at least two subject sites, said calibration measurement comprising the steps of
b1) measuring pressure at a branching point between the inspiratory conduits throughout oscillation;
b2) measuring individual flows at the subject sites with the pulmonary ventilation measuring devices throughout oscillation;
c) calculating calibration impedances for each inspiratory pathway as a frequency domain ratio of the pressure at the branching point over the corresponding flow at the subject site.
d) populating the subject sites with subjects;
e) performing a measurement manoeuvre by providing oscillatory gas flow from the controllable flow source to at least two subject sites, said measurement comprising the steps of
e1) measuring pressure at a branching point between the inspiratory conduits throughout oscillation;
e2) measuring individual flows at the subject sites with the pulmonary ventilation measuring devices throughout oscillation;
f) calculating individual impedances for each subject according to the following formula:
$${Z}_{tr , k} = \frac{{P}_{insp}}{{V}_{k}} - {Z}_{calk}$$
wherein Z_{tr,k} is a transfer impedance of the subject at site k, Pᵢₙₛₚ is a pressure at the branching point, Vₖ is a calibration flow obtained from the flow measurement device at site k and Z_{cal,k} is i a calibration impedance of a given pathway.

39. The method of claim 38, wherein the oscillatory gas flow in steps b) and e) is controlled to reproduce a predetermined flow rate, volume or pressure waveform.

40. The method of claim 39, wherein the waveform varies at a single frequency or a mix of frequencies.
